(19) United States
(12) Patent Application Publication
(10) Pub. No.: US 2009/0264733 A1
(43) Pub. Date: Oct. 22, 2009

(54) MRI CONTRAST USING HIGH TRANSVERSE RELAXATION RATE CONTRAST AGENT

(75) Inventors: Curtis A. Corum, Shoreview, MN (US); Michael G. Garwood, Medina, MN (US); Djaudat S. Idiyatullin, Minneapolis, MN (US); Steen Moeller, St. Louis park, MN (US)

Correspondence Address: SCHWEGMAN, LUNDBERG & WOESSNER, P.A., P.O. BOX 2938, MINNEAPOLIS, MN, 55402, US

(73) Assignee: Regents of the University of Minnesota, St. Paul, MN (US)

(21) Appl. No.: 12/425,113

(22) Filed: Apr. 16, 2009

Related U.S. Application Data
(60) Provisional application No. 61/045,927, filed on Apr. 17, 2008.

(51) Int. Cl. A61B 5/055

(52) U.S. Cl. 600/420

(57) ABSTRACT

This document discloses, among other things, a system and method of creating a positive contrast magnetic resonance imaging (MRI) feature using a high transverse relaxation rate contrast agent.

### CLAIM OF PRIORITY

This patent application claims the benefit of priority, under 35 U.S.C. Section 119(e), to Curtis A. Corum et al, U.S. Provisional Patent Application Ser. No. 61/045,927, entitled “POSITIVE CONTRAST MRI USING HIGH TRANSVERSE RELAXATION RATE CONTRAST AGENT,” filed on Apr. 17, 2008 (Attorney Docket No. 00600.721PRV), which is incorporated herein by reference.

### STATEMENT REGARDING FEDERALLY SPONSORED RESEARCH OR DEVELOPMENT

This invention was made with government support under award number BTRR P 41 RR008079 from the National Institutes of Health (NIH). The government has certain rights in this invention.

### BACKGROUND

Images can be taken of the body, or certain portions or aspects thereof, using various medical imaging techniques, such as magnetic resonance imaging (MRI), computed tomography (CT), angiography, etc. There exists a need to improve certain aspects thereof.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 includes a magnetic resonance system according to one example.

FIGS. 2A, 2B, and 2C include diagrams for a pulse sequence for SWIFT according to one example.

FIG. 3 includes a flow chart of a method according to one example.

FIGS. 4A-4F illustrates selected images of a mouse brain.

### DETAILED DESCRIPTION

The present inventors have recognized, among other things, a system and method for magnetic resonance (MR) contrast using contrast agents having high transverse relaxation rates. In one example, an MR image (MRI) having positive contrast can be generated. In general, positive contrast appears as a bright region in an image and refers to shortened relaxation times on T1-weighted images. Negative contrast, on the other hand, reduces signal intensity and appears as a dark region in an image.

Part 1 includes a description of a magnetic resonance system. Part 2 includes a description of the SWIFT, UTE, and BLAST methodologies. Part 3 includes a description of methods for using a contrast agent.

### Part 1

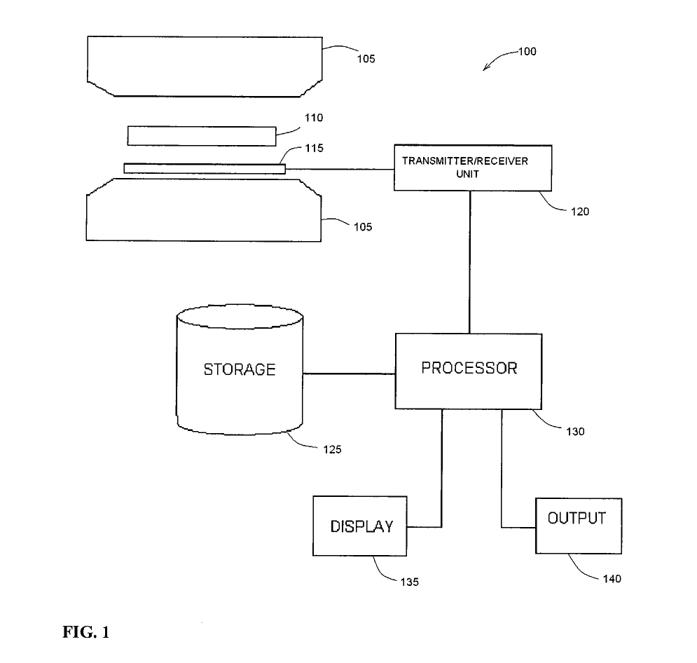

FIG. 1 includes a block diagram of magnetic resonance system 100. System 100, or selected parts thereof, can be referred to as an MR scanner.

Magnetic resonance system 100, in one example, depicts an imaging system 100 having magnet 105. In one example, system 100 includes an electron paramagnetic resonance system. Magnet 105 can provide a biasing magnetic field. Coil 115 and subject 110 are positioned within the field of magnet 105. Subject 110 can include a human body, an animal, a phantom, or other specimen. Coil 115, sometimes referred to as an antenna, can include a transmit coil, a receive coil, a separate transmit coil and receive coil, or a transceiver coil. Coil 115 is in communication with transmitter/receiver unit 120 and with processor 130. In various examples, coil 115 both transmits and receives radio frequency (RF) signals relative to subject 110. Transmitter/receiver unit 120 can include a transmit/receive switch, an analog-to-digital converter (ADC), a digital-to-analog converter (DAC), an amplifier, a filter, or other modules configured to excite coil 115 and to receive a signal from coil 115. Transmitter/receiver unit 120 is coupled to processor 330.

Processor 130 can include a digital signal processor, a microprocessor, a controller, or other module. Processor 130, in one example, is configured to generate an excitation signal (for example, a pulse sequence) for coil 115. Processor 130, in one example, is configured to perform a post-processing operation on the signal received from coil 115. Processor 130 is also coupled to storage 125, display 135 and output unit 140.

Storage 125 can include a memory for storing data. The data can include image data as well as results of processing performed by processor 130. In one example, storage 125 provides storage for executable instructions for use by processor 130. The instructions can be configured to generate and deliver a particular pulse sequence or to implement a particular algorithm.

Display 135 can include a screen, a monitor, or other device to render a visible image corresponding to subject 110. For example, display 135 can be configured to display a radial projection, a Cartesian coordinate projection, or other view corresponding to subject 110. Output unit 140 can include a printer, a storage device, a network interface or other device configured to receive processed data.

### Part 2

In nuclear magnetic resonance (NMR, also abbreviated as magnetic resonance, MR), RF excitation can be described as sequential, simultaneous, and random. Three different corresponding NMR techniques are used, including continuous wave (CW), pulsed, and stochastic.

Pulsed FT spectroscopy can be used with high resolution NMR. MRI has additional technical requirements over high resolution NMR. Because the objects of interest are much larger than a test tube, inevitably the static and RF fields used in MRI are more inhomogeneous than those used in high resolution NMR.

As in CW, the SWIFT method uses RF sweep excitation and uses a sweep rate that exceeds the sweep rate of the CW method by more than a few orders of magnitude. Unlike the CW method in which the signal is acquired in the frequency domain, in SWIFT, the signal is considered as a time function, as in the pulsed FT method. In addition, SWIFT uses the correlation method similar to stochastic NMR in order to extract proper spectral information from the spin system response.

The rapid-scan FT technique and SWIFT technique have some common properties but are different in point of view to system response on excitation. Rapid-scan FT considers the system response in frequency domain and SWIFT considers the system response in the time domain. As a result, the spectra obtained using SWIFT is insensitive to the linearity of the sweep rate. This permits use of a broad class of frequency modulated pulses having more uniform excitation profiles than the chirp excitation required in rapid-scan FT. SWIFT also provides virtually simultaneous excitation and acquisition of signal. Accordingly, SWIFT has a “zero echo time”, and so is well-suited for studying objects having very fast spin-spin (or transverse) relaxation (or very short T2). SWIFT allows analysis of R₁ (T₁) while substantially mitigating the effects of R₂. SWIFT can be used for MRI of quadrupolar nuclei, such as sodium-23, potassium-39, and boron-11.

According to one example, a short T₂ (or T₂*) value is less than approximately 5 ms. As such, a T₂ value of less than 5 ms may present difficulties for gradient-echo (GRE) magnetic resonance imaging. A particularly short T₂ (or T₂*) value is less than, for example, 1 ms. On the other hand, SWIFT can be used for MR imaging with a T₂ (or T₂*) value shorter than 5 ms or shorter than 1 ms. Stated differently, an R₂ (or R₂*) greater than 200 is considered large and a particularly large value would be greater than 1,000.

### SWIFT Methodology

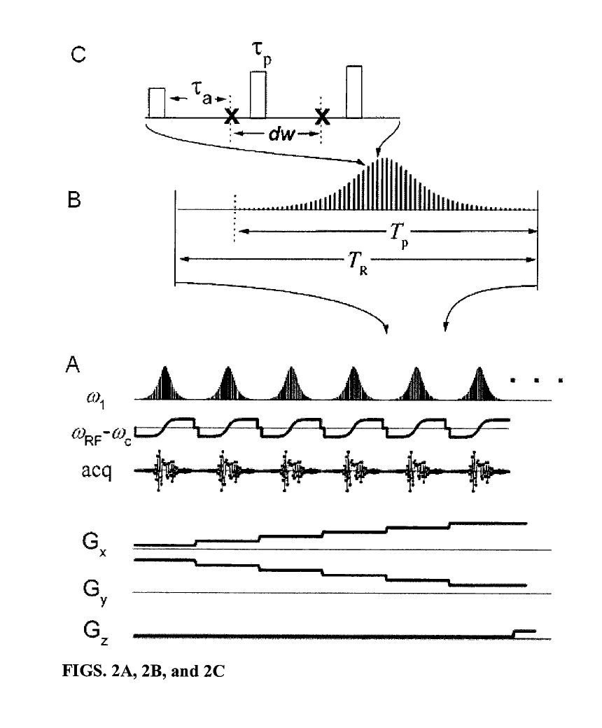

SWIFT can be modeled by the method presented in FIG. 2A. SWIFT employs a sequence of frequency-modulated pulses with short repetition time T_{R} that exceeds the pulse length T_{P} by at least the amount of time needed for setting a new value (or orientation) of a magnetic field gradient used to encode spatial information. The images are processed using 3D back-projection reconstruction. In one example, frequency-modulated pulses from the hyperbolic secant family (HSn pulses) are used. In FIG. 2B, one shaped pulse is represented which includes N different sub-pulse elements with time-dependent amplitudes and phases. During the FM pulse, an isochromat follows the effective RF field vector until the instant resonance is attained. At resonance, the isochromat is released from the RF pulse's “hug” and thereafter almost freely precesses with a small decaying modulation, yielding spectral contamination. Thus, to extract spectral information from such a spin system response, processing is performed using a cross-correlation method similar to the method of recovering phase information in stochastic NMR. The theoretically achievable signal-to-noise ratio (SNR) per unit time for SWIFT for TR<<T1 is the same as that for pulsed FT. During SWIFT acquisition, the applied imaging gradients usually exceed all intrinsic gradients due to susceptibility or inhomogeneity. For this condition the images obtained are fully independent of transverse relaxation and signal intensity depends only on T1 and spin density. The maximum T₁ contrast depends on effective flip angle and the best compromise between sensitivity and contrast will have flip angles exceeding two times the Ernst angle. If flip angles are very small, T₁ contrast is negligible, and contrast comes entirely from spin density. Other kinds of contrast can be reached by an appropriate preparation sequence prior to or interleaved with the image acquisition.

SWIFT provides novel and beneficial properties for MRI, including the following:

(a) fast: SWIFT eliminates the delays associated with refocusing pulses or gradient inversion, and also time for an excitation pulse, which is integrated with the acquisition period. As in other fast imaging sequences, SWIFT is limited by existing imaging system hardware and chosen compromise between acquisition speed, spatial resolution and SNR.

(b) sensitive to short T₂: SWIFT is sensitive to excited spins having T₂>1/SW (SW=spectral width). To be specifically resolved, T₂>N/SW must be satisfied, which is theoretically feasible even for solid objects by increasing SW.

(c) reduced motion artifacts: Because SWIFT has no “echo time” it is less sensitive to motion artifacts. It loses less signal due to either diffusion in the presence of a gradient or uncompensated motion than other fast sequences.

(d) reduced dynamic range requirement: Because the different frequencies are excited sequentially the resulting signal is distributed in time with decreased amplitude of the acquired signal. This allows more effective utilization of the dynamic range of the digitizer.

(e) quiet: SWIFT uses a small step when changing gradients between projections, and thus, fast gradient switching that creates loud noise can be avoided. SWIFT can also be operated in rapid updated mode to reach high temporal resolution in dynamic studies. This pseudo-temporal resolution is possible because projection reconstruction, unlike Fourier imaging, samples the center of k-space with every acquisition.

### UTE Methodology

An ultrashort echo-time (UTE) pulse sequence can be used for imaging tissues or tissue components having a small value spin-spin (transverse) relaxation time T₂. With UTE imaging, the radio frequency pulse duration is of the order T₂ and the rotation of tissue magnetization into the transverse plane is incomplete. Typically, UTE entails rapid data acquisition and a TE can be approximately 0.08 ms. An example of a UTE pulse sequence includes a half excitation pulse and radial imaging from the center of k-space. Variations of UTE can be tailored to suppress fat or for imaging short or long T₂ components.

### BLAST Methodology

A pulse sequence described as Back-projection Low Angle ShoT (BLAST) can be used for fast imaging of liquids and solids. BLAST is typically associated with imaging of animals and is based on low angle pulse excitation in combination with back-projection reconstruction. BLAST, like UTE and like SWIFT, is suitable for use in imaging tissue or components having short T₂.

### Part 3

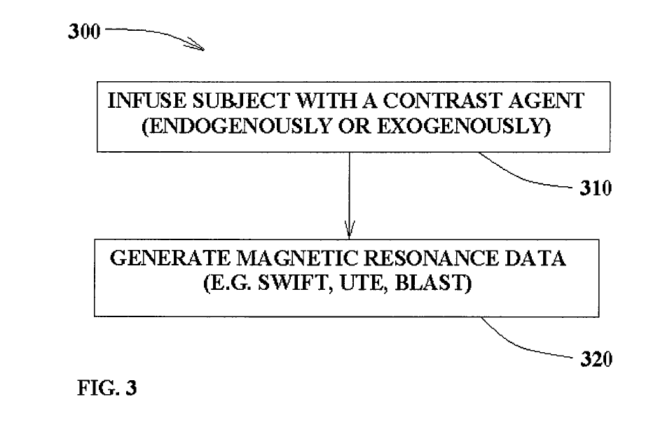

FIG. 3 includes a flow chart of method 300 according to one example. Method 300 can be used to generate a magnetic resonance (MR) image or MR image features having positive contrast enhancement. At 310, method 300 includes infusing a subject with a contrast agent. Infusion can be performed endogenously in which a naturally occurring agent can serve as the contrast agent. For example, iron content that accumulates in the liver of a subject can be used as the contrast agent. In addition, the contrast agent can be infused exogenously. For example, an orally administered or intravenously administered contrast agent can be delivered to the subject.

At 320, method 300 includes generating magnetic resonance data. The data can be generated using a pulse sequence or protocol having a short dead time. Examples of MR protocols can include SWIFT, UTE, and BLAST.

Consider an example of method 300 using an in-vivo wild type mouse brain.

As shown in the figure, at 310, the contrast agent can injected. In one example, the contrast agent includes an intravenously administered bolus of mono-crystalline ion oxide nano-particle solution, sometimes referred to as MION-47. MION-47 has both R₁ and R₂* relaxivity, but the R₂* relaxivity typically dominates at high concentrations and/or high fields (e.g., the R1 and R2 values are approximately 29 and 60 mMsec-1 at 0.47 T, 39° C.).

At 320, a magnetic resonance (MR) technique is used to generate data for the subject. An example of an MR technique includes SWeep Imaging with Fourier Transform (SWIFT). High field and high concentration R₁ induced positive T₁ contrast can be generated using a MION-47 dose of 5 mg/kg and 20 mg/kg.

As noted elsewhere in this document, SWIFT is a novel radial imaging sequence utilizing gapped frequency-swept pulse excitation and nearly simultaneous signal acquisition in the dead time between the gaps. SWIFT utilizes the correlation method which removes phase differences due to the time of excitation and produces free induction decay (FID) data as if the spins were simultaneously excited by a short duration pulse. SWIFT has an intrinsically short dead-time, for example, ˜5-15 μs. SWIFT, as with UTE and BLAST, provides good sensitivity to very fast relaxing spins (short T₂ or T₂*).

In one example, a wild type mouse (20 g) can be anesthetized with 2% Isoflurane, catheterized with pre-loaded line of 10:1 dilution of MION-47 and placed in a 9.4 T 31 cm bore animal magnet. The animal can be placed in a heated holder and quadrature surface coil with two ˜1 cm loops at a location about 2 mm from the top of the head. A syringe reservoir of 0.5 cc of the diluted MION-47 can terminate the I.V. line. A pre-injection series of SWIFT images can be acquired during an initial 20 minute segment, and then MION solution can be injected slowly by hand to 5 mg/kg dose (100 μL). After approximately 10 minutes, a post contrast series of SWIFT images can be taken over a duration lasting approximately 30 minutes, and then another 300 μL bolus of MION dilution can be injected for a total dose of 20 mg/kg. Post second bolus imaging was acquired during next 30 minutes.

One example of a method allows T₁ weighted imaging in the presence of large R₁ relaxation. In the following, S denotes the system, S₀ denotes the thermal equilibrium magnetization signal, and R₂** (sometimes referred to as R₂^{†} or R₂ dagger) denotes inhomogeneity.

For example:

S=S₀(1−exp(−T_{R}/T₁))exp(−T_{E}/T₂*)
T₂*=1/R₂*
R₂*=R₂+R₂**
T₁=1/R₁

In SWIFT, the T_{E}˜=0, so the contrast becomes:

S=S_{SS}(1−exp(−T_{R}/T₁))

A typical non-short T₂ sensitive sequence has exp(−T_{E}/T₂*)=0 when R₂*=1/T₂* is large.

In the example described herein, a series of images summarize the results. In the image datasets, the bandwidth (for excitation of base-band and acquisition) is 62.5 kHz. Each 3D radial SWIFT dataset includes 32,000 unique FID views (spokes). Duty cycle used for excitation Hyperbolic Secant pulse was 25%. TR was 6.1 ms with 4.1 ms of acquisition time included. The diameter of field of view (FOV) was 3 cm. Total time for each image was 3.5 minutes including steady state scans. Processing of the SWIFT data was accomplished by correlation with the RF shape file, and data driven RF distortion correction. The radial reconstruction was accomplished by gridding with 1.25× over-sampled width 2.5 Kaiser-Bessel kernel and 1/r² density weighting.

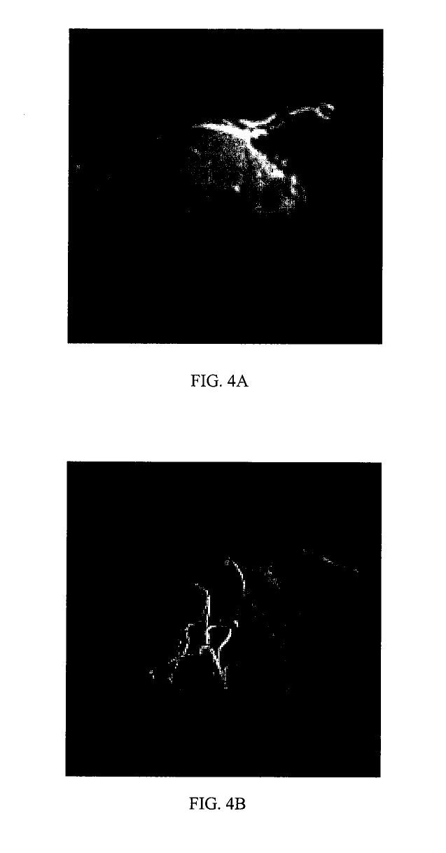

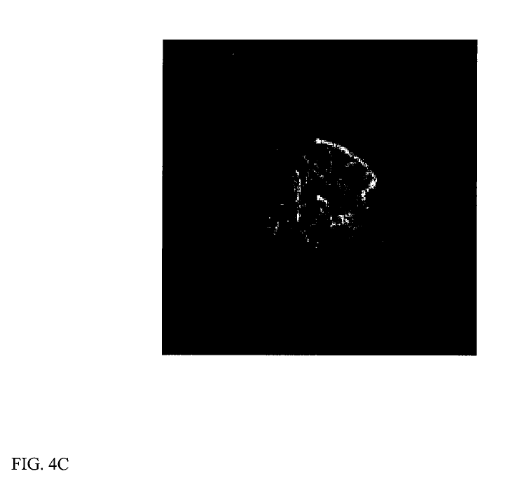

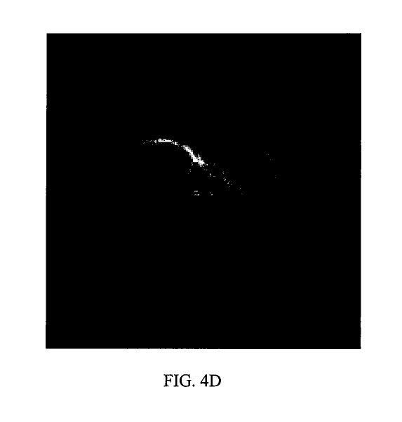

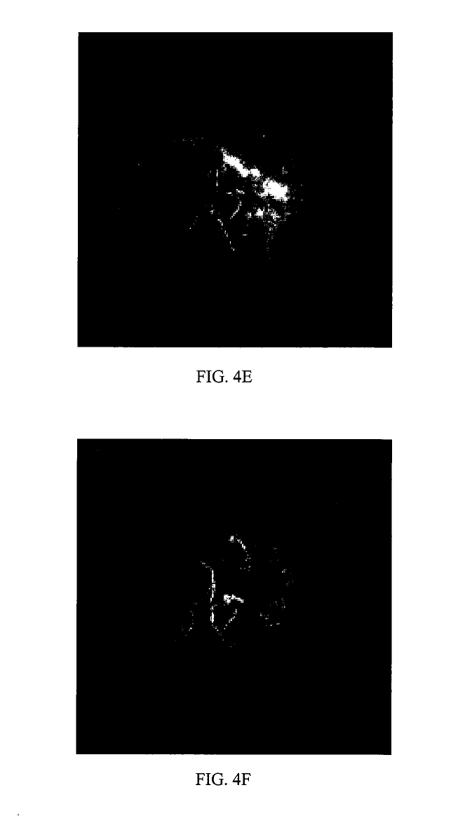

FIG. 4A illustrates generally an example of a representative slice from the pre-MION 11° flip dataset. In the MR images, the mouse is slightly rotated transverse.

FIG. 4B illustrates generally an example of a maximum intensity projection (MIP) of the pre-MION 45° flip dataset. Inflow contrast appears in the large arteries.

FIG. 4C illustrates generally an example of a subtraction, and the MIP of the 11° flip, 5 mg/kg MION—Pre-MION datasets. Enhancement appears in the largest veins and is not likely to be a result of inflow (due to being venous and the image being a subtraction).

FIG. 4D illustrates generally an example of a 45° flip, 5 mg/kg MION—Pre-MION MIP; enhancement appears in the medium and large veins, and some arteries.

FIG. 4E illustrates generally, after the second bolus, an example of a 45° flip, 20 mg/kg MION—Pre-MION MIP. In this example, contrast appears throughout the vascular system, and blooming but no signal loss in large veins.

FIG. 4F illustrates generally an example of a different enhancement pattern, with less blooming, obtained by subtraction the two post injection datasets, e.g., 20 mg/kg MION—5 mg/kg MION MIP.

Positive T₁ contrast with Fe nanoparticles in a wild type mouse brain, in-vivo, can be generated using the present subject matter.

In certain examples, this system or method can allow for positive contrast (bright, hyperintense, or enhancing) image features to be obtained from contrast agents that with existing MRI methods would yield negative contrast (dark, or hypointense) image features. A short T₂ sensitive method such as SWIFT, Ultra-short Time of Echo (UTE), or Back-projection Low-Angle shot (BLAST) MRI can be performed before, during, or after injection of a bolus or infusion of contrast agent. The contrast agent can produce a significantly higher R₂ (transverse relaxation rate constant) value than Gd based agents and still yield positive contrast (enhancement) in the tissues or organs of interest.

In an example, the contrast agents can include an iron (Fe) based contrast agent (e.g., a mono-crystalline ion oxide nano-particle solution such as MION-47), a manganese (Mn) based contrast agent, a dysprosium (Dy) based contrast agent, or other contrast agent. In certain examples, the contrast agent can include a small particle iron oxide (or superparamagnetic iron oxide) (SPIO) (e.g., Ferridex, or Ferrum oxide), ultra small particle iron oxide (or ultrasmall superparamagnetic iron oxide) (USPIO), labeled, unlabeled, or other contrast agents.

In certain examples, the contrast agents can include one or more contrast agent illustrated in Table 1, below.

TABLE 1

| | CENTRAL | | | | TRADE |
|---|---|---|---|---|---|
| NAME OF COMPOUND | MOIETY | RELAXIVITY | DISTRIBUTION | INDICATION | MARK |
| Gadopentate dimeglumine, | Gd3+ | r1 = 3.4, | intravascular, | neuro/whole | Magnevist |
| Gd-DTPA | | r2 = 3.8, | extracellular | body | |
| | | B0 = 1.0 T, | | | |
| | | Xm = 2.7 10-2 | | | |
| Gadoterate meglumine, | Gd3+ | r1 = 3.4, | intravascular, | neuro/whole | Dotarem |
| Gd-DOTA | | r2 = 4.8, | extracellular | body | |
| | | B0 = 1.0 T, | | | |
| | | Xm = 2.7 10-2 | | | |
| Gadodiamide, Gd-DTPA- | Gd3+ | r1 = 3.9, | intravascular, | neuro/whole | Omniscan |
| BMA | | r2 = 4.3, | extracellular | body | |
| | | B0 = 1.0 T, | | | |
| | | Xm = 2.7 10-2 | | | |
| Gadoteridol, Gd-HP-DO3A | Gd3+ | r1 = 3.7, | intravascular, | neuro/whole | Prohance |
| | | r2 = 4.8, | extracellular | body | |
| | | B0 = 1.0 T, | | | |
| | | Xm = 2.7 10-2 | | | |
| Gadob gastrointestinal | bowel | Phase III, | Gadolite 60 | | |
| | marking | oral | | | |
| | | suspension | | | |
| MnCl 2 | Mn2+ | paramagnetic | gastrointestinal | bowel | Lumenhance |
| | | | | marking | |
| Fatty emulsion | fatty liquid | short T1- | gastrointestinal | bowel | |
| | | relaxation | | marking | |
| | | time | | | |
| Vegetable oils | fatty liquid | short T1- | gastrointestinal | bowel | |
| | | relaxation | | marking | |
| | | time | | | |
| Sucrose polyesters | fatty liquid | short T1- | gastrointestinal | bowel | |
| | | relaxation | | marking | |
| | | time | | | |
| Mangafodipir trisodium | Mn2+ | r1 = 2.3, | hepatobiliary, | liver lesions | Teslascan |
| MN-DPDP, Managnese | | r2 = 4.0, | pancreayiv, | | |
| dipyroxyl diphosphate | | B0 = 1.0 T | adrenal | | |
| Gadobenate di- | Gd3+ | r1 = 4.6, | intravascular, | neuro/whole | Multihance |
| meglumine, Gd-BOPTA | | r2 = 6.2, | extracellular, | body, liver | |
| | | B0 = 1.0 T | hepatobiliary | lesions | |
| Gadoxetic acid, Gd-EOB- | Gd3+ | short T1- | hepatobiliary | liver lesions | Eovist |
| DTPA | | relaxation | | | |
| | | time | | | |
| Fe-HBED | Fe2+ | | hepatobiliary | liver lesions | |
| Fe-EHPD | Fe2+ | | hepatobiliary | liver lesions | |
| Liposomes, paramagnetic | Gd3+ | | RES-directed | liver lesions | |
| Mn-EDTA-PP (liposomes) | Mn2+ | r1 = 37.4, | | | Memosomes |
| | | r2 = 53.2, | | | |
| | | B0 = 0.5 T | | | |
| Polylysine-(Gd-DTPA)x- | Gd3+ | | lymph nodes | staging of | |
| dextran | | | | lymph | |
| | | | | nodes | |
| Diphenylcyclohexyl | Gd3+ | | intravascular, | MR- | |
| phosphodiester-Gd-DTPA, | | | short elimination | angiography, | |
| MS 325 EPIX | | | half life | vasc. | |
| | | | | capillary | |
| | | | | permeability | |
| MP 2269, 4-pentyl-bicyclo | Gd3+ | r1 = 6.2, | intravascular | MR- | |
| [2.2.2] octan-1-carboxyl-di- | | B0 = 1.0 T | | angiography | |
| L-aspartyllysine-DTPA | | | | | |
| (Gd-DTPA)-17, 24 | Gd3+ | r1 = 11.9, | intravascular | MR- | Gadomer-17, |
| cascade polymer | | B0 = 1.0 T, | | angiography | 24 |
| | | (r2 = 16.5) | | vascularis | |
| Gd-DTPA-PEG polymers | Gd3+ | r1 = 6.0, | intravascular | MR- | |
| (polyethylene glycol) | | B0 = 1.0 T | | angiography | |
| | | | | vascularis, | |
| | | | | capillary | |
| | | | | permeability | |
| (Gd-DTPA)n-albumin, (Gd- | Gd3+ | r1 = 14.4, | intravascular | MR- | |
| DOTA)n-albumin | | B0 = 0.23 T | | angiography | |
| | | | | vascularis, | |
| | | | | MR- | |
| | | | | mammography | |
| (Gd-DTPA)n-polylysine | Gd3+ | r1 = 13.1, | intravascular | MR- | |
| | | B0 = 0.23 T | | angiography | |
| (Gd-DTPA)n-dextran | Gd3+ | | intravascular | MR- | |
| | | | | angiography | |
| Manganese substituted | Mn2+ | r1 = 21.7, | intravascular | MR- | |
| hydroxylapatite PEG-APD | | r2 = 26.9, | | angiography | |
| (MnHA/PEG-APD) | | B0 = 1.0 T | | | |
| WIN 22181 | Gd3+ | r1 = 9.5 | | MR- | |
| | | | | urography | |
| Sprodyamide, Dy-DTPA- | Dy2+ | T2*enhanced, | intravascular | blood flow | |
| BMA | | r1 = 3.4, | | perfusion | |
| | | r2 = 3.8, | | | |
| | | B0 = 0.47 T, | | | |
| | | Xm = 4.46 102 | | | |
| Dy-DTPA | Dy2+ | T2*enhanced, | intravascular | blood flow | |
| | | Xm = 4.8 102 | | perfusion | |
| Albumin-(Dy-DTPA)x | Dy2+ | T2*enhanced | intravascular | blood flow | |
| | | | | perfusion | |
| Ferrum oxid. (USAN), | Fe2+/Fe3+ | r1 = 40.0, | RES-directed | liver lesions | Endorem, |
| SPIO, Ami-25, dextran- | | r2 = 160, | | and control | Feridex |
| coated | | B0 = 0.47 T, | | | |
| | | Xm = 0.4 | | | |
| Ferrixan, Carboxy-dextran | Fe2+ | r1 = 25.4, | RES-directed | liver lesions | Resovist |
| coated iron oxide | | r2 = 151 | | | |
| nanoparticles, SHU 555A | | | | | |
| USPIO, AMI-227 | Fe3+/Fe2+ | r1 = 25, | vascular, lymph | MR- | Sinerem, |
| | | r2 = 160, | v. hepatocyte | angiography | Combidex |
| | | B0 = 0.47 T, | (AG-USPIO) | vascular | |
| | | Xm = 0.34, | | staging of | |
| | | r1 = 23.3, | | RES- | |
| | | r2 = 48.9, | | directed | |
| | | B0 = 0.47 T | | liver | |
| | | | | diseases | |
| Fe O-BPA USPIO | Fe3+/Fe2+ | | vascular | MR- | |
| | | | | angiography | |
| MION, Monocrystalline | Fe3+/Fe2+ | r1 = 3.7, | vascular lymph | MR- | |
| iron oxide nanoparticles | | r2 = 6.5, | v. (MION-46) | angiography, | |
| | | B0 = 0.47 T, | tumours, FAB- | MR- | |
| | | Xm = 0.11 | MION, | lymphography, | |
| | | | antimyosin, FAB- | tumour | |
| | | | MION | detection, | |
| | | | | infarction | |
| Magnetic starch | Mr 2+/Mr 3+ | r1 = 27.6, | RES-directed | liver | |
| microspherers | | r2 = 183.7, | | lesions, | |
| | | B0 = 1.0 T | | spleen | |
| PION, polycrystalline iron | Fe2+/Fe3+ | T2*enhanced, | RES-directed | liver | |
| oxide nanoparticles (larger | | r2/r1 = 4.4, | lymph v. | lesions, MR | |
| particles = DDM 128, | | r2/r1 = 7 | hepatocyte | lymphography | |
| PION-ASF) | | | | | |
| Ferromuxsilum (USAN) | Fe3+/Fe2+ | T2*enhanced, | gastrointestinal | bowel | Lumirem, |
| AMI-121 | | Xm = 0.23 | | marking | Gastromark |
| Ferristene (USAN) oral | Fe2+/Fe3+ | T2*enhanced | gastrointestinal | bowel | Abdoscan |
| magnetic particles (OMP) | | | | marking | |
| Perfluorooctylbromide | water | proton | gastrointestinal | bowel | Perflubron, |
| (PFOB) | immiscible | density | | marking | Imagent R, GI, |
| | liquid | reduction, | | | USA |
| | | signal void | | | |
| Barium suspensions and | Ba3+, | diamagnetic, | gastrointestinal | bowel | various |
| clay mineral particles OMP | Al3+, Si2+ | T2-short | | marking | mixtures |
| Dy-tatraphenyl-porphyrin | Dy2 + Ho2+ | high | tumour selective | tumour | |
| sulfonate, Dy-TPPS or Ho- | | susceptibility | uptake | detection | |
| TPPS | | | | and control | |

In an example, the present subject matter can include magnetic resonance (MR) clinical dynamic contrast enhancement (DCE) imaging, Magnetic Resonance Angiography (MRA), or other T1 weighted contrast enhanced MRI.

The present system and method allows the use of high R₂ agents, such as Fe based agents. An example of the present subject matter does not rely on a low R₂ value contrast agent such as those based on Gadolinium (Gd).

Gd based contrast agents may have side effects in some patients, such as Nephrogenic Systemic Fibrosis (NSF). The method embodied in this document allows many types of contrast agents (including non-Gd based agents) to be used for positive contrast MRI procedures. Iron (Fe) nanoparticle based agents can be used to attach multiple ligands (functionalization) and have additional degrees of freedom (such as size and shape) to fine tune the contrast mechanism. An example of the present subject matter does not rely on off-resonance pre-pulsing, intermolecular multiple quantum coherence or other filtering methods that may degrade the signal intensity and do not directly detect the T₁ weighted signal.

In one example, the MR scanner is configured to apply a longitudinal relaxation time (T₁) sensitive magnetic resonance imaging (MRI) pulse waveform on at least a portion of a subject and acquire a positive contrast image from the at least a portion of the subject. In an example, the acquired positive contrast image results from interaction with a contrast agent having a high transverse relaxation rate (R₂).

In other examples, one or more other system or method described herein can be implemented using an MR scanner, or one or more other component, such as a processor or other controller, a memory device, a display, or other device configured to assist in acquiring a positive contrast image from at least a portion of a subject.

In certain examples, the system or method described herein can be implemented using a set of instructions in a computer-readable medium or machine readable medium.

In certain examples, the contrast agent can have both R₁ and R₂ (and R₂*) relaxivity, but can be quantified predominantly by R₁ relaxivity. Further, the contrast agent can be injected into the vasculature, can be injected into a tissue, can be expressed as part of a reporter gene system, or can be intrinsic to a tissue (e.g., including Iron containing plaques). In other examples, one or more cell can be labeled with the contrast agent or be injected.

Further, any bodily tissue can be imaged using the system or method disclosed herein, generally including the vasculature (e.g., using angiography, in any part of the body), or including, but not limited to, the brain, the heart, the extremities, the liver, the kidney, the spleen, etc. In certain examples, the liver, the spleen, or other high natural Fe content tissue can be analyzed.

In an example, cancer in any part of the body can be analyzed suing the system or method disclosed herein, including the brain, breast, lung, liver, bone, etc. In certain examples, this can be accomplished utilizing pre-contrast and post-contrast subtraction, or dynamic (pre-contrast and multiple post-contrast time course images), or perfusion imaging using pre and one or more post-contrast timed doses.

### Some Notes

The above detailed description includes references to the accompanying drawings, which form a part of the detailed description. The drawings show, by way of illustration, specific embodiments in which the invention can be practiced. These embodiments are also referred to herein as “examples.” All publications, patents, and patent documents referred to in this document are incorporated by reference herein in their entirety, as though individually incorporated by reference. In the event of inconsistent usages between this document and those documents so incorporated by reference, the usage in the incorporated reference(s) should be considered supplementary to that of this document; for irreconcilable inconsistencies, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In the appended claims, the terms “including” and “in which” are used as the plain-English equivalents of the respective terms “comprising” and “wherein.” Also, in the following claims, the terms “including” and “comprising” are open-ended, that is, a system, device, article, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

Method examples described herein can be computer-implemented at least in part. Some examples can include a computer-readable medium or machine-readable medium encoded with instructions operable to configure an electronic device to perform methods as described in the above examples. An implementation of such methods can include code, such as microcode, assembly language code, a higher-level language code, or the like. Such code can include computer readable instructions for performing various methods. The code can form portions of computer program products. Further, the code can be tangibly stored on one or more volatile or non-volatile computer-readable media during execution or at other times. These computer-readable media can include, but are not limited to, hard disks, removable magnetic disks, removable optical disks (e.g., compact disks and digital video disks), magnetic cassettes, memory cards or sticks, random access memories (RAM's), read only memories (ROM's), and the like.

The above description is intended to be illustrative, and not restrictive. For example, the above-described examples (or one or more aspects thereof) can be used in combination with each other. Other embodiments can be used, such as by one of ordinary skill in the art upon reviewing the above description. The Abstract is provided to comply with 37 C.F.R. §1.72(b), to allow the reader to quickly ascertain the nature of the technical disclosure. It is submitted with the understanding that it will not be used to interpret or limit the scope or meaning of the claims. Also, in the above Detailed Description, various features can be grouped together to streamline the disclosure. This should not be interpreted as intending that an unclaimed disclosed feature is essential to any claim. Rather, inventive subject matter can lie in less than all features of a particular disclosed embodiment. Thus, the following claims are hereby incorporated into the Detailed Description, with each claim standing on its own as a separate embodiment. The scope of the invention should be determined with reference to the appended claims, along with the full scope of equivalents to which such claims are entitled.

What is claimed is:

1. A method comprising:
infusing a subject with a contrast agent, the contrast agent having a high transverse relaxation rate (R₂); and
generating magnetic resonance data in which the contrast agent contributes to a reduced longitudinal relaxation time (T₁) in the subject.

2. The method of claim 1 wherein generating magnetic resonance data includes applying a gapped pulse sequence.

3. The method of claim 2 wherein applying the gapped pulse sequence includes applying a swept frequency excitation and further including substantially simultaneously acquiring a signal in a time shared mode.

4. The method of claim 1 wherein generating magnetic resonance data includes implementing at least one of ultra-short echo time (UTE) and back projection low angle shot (BLAST).

5. The method of claim 1, wherein the contrast agent has an R₂ value greater than a gadolinium (Gd) based contrast agent.

6. The method of claim 1, wherein the contrast agent includes iron (Fe).

7. The method of claim 6, wherein the contrast agent includes a formulated mono-crystalline ion oxide nano-particle solution.

8. The method of claim 1, wherein the contrast agent includes manganese (Mn).

9. The method of claim 1, wherein the contrast agent includes dysprosium (Dy).

10. The method of claim 1, wherein generating magnetic resonance data includes generating an image having positive contrast.

11. The method of claim 1, wherein generating magnetic resonance data in includes using a pulse sequence sensitive to short transverse relaxation times (T₂).

12. The method of claim 1, wherein infusing the subject with a contrast agent includes at least one of ingesting orally and introducing intravenously.

13. The method of claim 1, further including generating an image using the magnetic resonance data.

14. A method comprising:
applying a transverse relaxation time (T₂) magnetic resonance imaging (MRI) pulse waveform to a subject; and
acquiring data from the subject, wherein the data results from interaction with a contrast agent having a high transverse relaxation rate (R₂).

15. The method of claim 14, wherein applying the T₂ MRI pulse waveform includes applying at least one of a SWIFT pulse sequence, a UTE pulse sequence, and a BLAST pulse sequence.

16. The method of claim 14, wherein acquiring data includes acquiring data in a radial projection.

17. The method of claim 14, further including infusing the subject with the contrast agent.

18. The method of claim 17, wherein infusing the subject with the contrast agent includes infusing with a contrast agent having a high R₂ relaxation rate.

19. The method of claim 17, wherein infusing the subject with the contrast agent includes at least one of injecting into a vasculature structure of the subject, injecting into a tissue of the subject, expressing as part of a reporter gene system, and endogenously infusing a tissue of the subject.

20. The method of claim 17, further including generating an image from the data, the image having at least one positive contrast feature.

21. An apparatus comprising:
an MRI scanner configured to apply a longitudinal relaxation time (T₁) sensitive magnetic resonance imaging (MRI) pulse waveform to a subject and acquire a positive contrast image from the subject, the acquired positive contrast image resulting from interaction with a contrast agent having a high transverse relaxation rate (R₂).

22. The apparatus of claim 21, wherein the MRI scanner includes a processor configured to control the MRI pulse waveform.

23. The apparatus of claim 21, wherein the MRI scanner includes a memory configured to store data corresponding to the image.

24. A machine readable medium having executable instructions stored thereon for performing a method comprising:
exciting a subject with a magnetic resonance pulse, the subject including a contrast agent having a high transverse relaxation rate (R₂); and
acquiring magnetic resonance data from the subject.

25. The machine readable medium of claim 24, wherein exciting the subject with the magnetic resonance pulse includes exciting the subject with a pulse having a short dead time.

26. The machine readable medium of claim 24, wherein exciting the subject with the magnetic resonance pulse includes delivering at least one of a SWIFT pulse sequence, a UTE pulse sequence, and a BLAST pulse sequence.

27. The machine readable medium of claim 24, wherein acquiring magnetic resonance data from the subject includes generating an image having T₁ contrast.
